(19) United States
(12) Patent Application Publication
(10) Pub. No.: US 2012/0197320 A1
(43) Pub. Date: Aug. 2, 2012

(54) Foraminoplasty Device

(75) Inventor: Zoltan Bereczki, Tampa, FL (US)

(73) Assignee: Laser Spine Surgical Center, LLC, Tampa, FL (US)

(21) Appl. No.: 13/016,358

(22) Filed: Jan. 28, 2011

(51) Int. Cl. A61B 17/56

(52) U.S. Cl. 606/86 R

(57) ABSTRACT

Tools, kits and methods for conducting surgeries, such as minimally invasive foraminotomies are provided. The tools are designed to permit practitioners to approach sites in need of a foraminotomy from an anterior posterior angle and thus to remedy pain with decreased discomfort to the patient.

### FIELD OF THE INVENTION

The present invention pertains to the field of surgical devices and techniques.

### BACKGROUND OF THE INVENTION

Spinal pain is one of the most unpleasant types of pain that a person can experience. Because the spine contains nerves that provide communication between the brain and other parts of the body, when one tries to relieve spinal pain, one must be particularly careful not to unintentionally damage the spine itself, nerves contained therein or connected thereto, or any of the surrounding tissue or bone.

In order to treat spinal pain, researchers and clinicians have developed a number of options. These options include administering pain medication to relieve the symptoms of the pain, physical therapy, and surgery. These options may be used alone or in combination, and in consultation with a health care professional, a patient may develop a protocol that best suits his or her needs.

One of the most severe types of pain is the pain that is caused by compression of the spinal nerves, and one source of this pain can be compression of a spinal nerve root as it exits the spinal canal. The compression may be caused by bone, disc, scar tissue, or excessive ligament development and for example, result in a pinched nerve.

In order to alleviate this type of pain, surgeons have developed a procedure that is known as a foraminotomy. In this procedure, the surgeon enlarges the passageway where a compressed spinal nerve roots exits the spinal canal. This passageway is referred to as the neuroforamen. A neuroforamen is located on each of left and right sides of the spine between each pair of vertebrae.

As persons of ordinary skill in the art are aware, depending on the etiology of a person's pain, it may be necessary to perform a foraminotomy in conjunction with a laminotomy or a laminectomy. Both the laminotomy and the laminectomy are spinal procedures that involve removing the lamina. In a laminotomy there is only partial removal of the lamina. In a laminectomy, all of the lamina is removed.

All surgeries, including foraminotomies carry with them some risk to a patient's health, and it is desirable to minimize both this risk and the discomfort of patients during and after surgery. To those ends, many surgeons have adopted minimally invasive surgical techniques.

Minimally invasive surgical techniques involve the use of tools that can be operated through relatively small incisions in a patient. In currently known techniques, surgeons can, for example, perform a foraminotomy without creating a large opening in the patient, i.e., they don't need to perform open back surgery. Using these techniques, surgeons have for a number of years addressed issues such as spinal stenosis by approaching the spine from the top of a bone and cutting away at both tissue and bone that impinge on a nerve root.

Unfortunately, even these minimally invasive techniques and the tools used to carry them out are not desirable for all patients. Therefore, patients and surgeons would benefit from the development of new tools and techniques. The present invention is directed to these issues.

### SUMMARY OF THE INVENTION

In various embodiments, the present invention is directed to novel and non-obvious tools, the use of these tools, and methods that provide means by which to perform foraminotomies and other surgeries. Through the use of these tools, practitioners may more effectively treat patients who suffer from pain.

According to a first embodiment, the present invention is directed to a surgical tool that may be used as a foraminotomy sleeve. This surgical tool comprises: (i) a sleeve, wherein the sleeve forms a first tube and has a first end and a second end, wherein the second end comprises an elongated section, wherein the elongated section is a continuation of less than all of the walls of the first tube; (ii) a port, wherein the port comprises a second tube that is connected to the first tube; and (iii) a handle, wherein the handle is configured to enable the surgical tool to be positioned or to be rotated along an axis of the sleeve when an appropriate force is applied. Both the first tube and the second tube are open at each of their ends. In some embodiments, the handle and port are each part of, connected to or extensions from an adapter that is located at the first end of the sleeve.

According to a second embodiment, the present invention is directed to a surgical tool that may be used as a stylus. This surgical tool comprises: (i) a tube, wherein the tube has a first end and a second end, wherein the second end comprises an elongated section, wherein the elongated section is a continuation of less than all of the walls of the tube; and (ii) an adapter, wherein the adapter is located at the first end of the tube and the adapter is wider than the tube.

According to a third embodiment, the present invention is directed to a surgical tool that may be used as a cutter. This surgical tool may also be referred to as a foraminotomy trephine and comprises: (i) a tube, wherein the tube has a first end and a second end, wherein the second end is serrated; and (ii) a handle, wherein the handle is connected to the tube and is configured to cause the surgical tool to rotate when an appropriate force is applied.

According to a fourth embodiment, the present invention is directed to a surgical tool that may be used as a soft tissue dilator. This surgical tool comprises: (i) a handle; and (ii) a longitudinal element comprising a tube, wherein the handle is located at a first end of the longitudinal element. The tube has at least one opening. This opening is located at a second end of the longitudinal element. The tube may be either closed or open at the opposite end, the first end. At the second end of the longitudinal element the outer surface is tapered.

According to additional embodiments, the present invention also provides kits. The kits may comprise any one or more of the aforementioned surgical tools and may also or alternatively comprise additional surgical tools, such as suction tools.

According to still further embodiments, the present invention provides methods for performing surgery. The methods may make use of any one or more of the aforementioned surgical tools. These methods may for example, be used in minimally invasive surgeries such as foraminotomies.

According to one of these methods, a practitioner: (i) inserts a foraminotomy sleeve into a patient, wherein the foraminotomy sleeve comprises an elongated section; (ii) causes the elongated section to move a nerve away from tissue or bone; (iii) inserts a cutter into a foraminotomy sleeve and cuts the tissue away from which the nerve was moved; and (iv) removes the tissue or bone. In some embodiments, prior to inserting the foraminotomy sleeve, the practitioner inserts a guide pin, and then a soft tissue dilator. The practitioner may then remove the soft tissue dilator.

After removing the soft tissue dilator, the practitioner may insert the foraminotomy sleeve over the guide pin, then insert a stylus over the guide pin, but within the foraminotomy sleeve. Subsequently, the guide pin and the stylus may be removed either together or one at a time.

The cutter may also be designed to enable the practitioner to irrigate the site of interest and to apply suction while the cutter is within the foraminotomy sleeve. To facilitate this step, an irrigation coupler may be used.

Through the use of the tools, kits, techniques and methods of the present invention, one may perform surgery in a manner that may have one or more of the following benefits, including causing less discomfort to a patient, being faster to perform, being more efficient, and causing less trauma.

### BRIEF DESCRIPTION OF THE FIGURES

FIGS. 1A and 1B are representations of a foraminotomy sleeve of an embodiment of the present invention.

FIGS. 2A and 2B are representations of a stylus of an embodiment of the present invention.

FIGS. 3A, 3B and 3C are representations of a cutter of an embodiment of the present invention.

FIGS. 4A, 4B and 4C are representations of an irrigation coupler of an embodiment of the present invention.

FIG. 5 is a representation of an irrigation coupler and a foraminotomy trephine assembly of an embodiment of the present invention in which the irrigation coupler and the foraminotomy trephine are engaged.

FIGS. 6A and 6B are representations of a cannulated dilator with a “T” handle of an embodiment of the present invention.

FIGS. 7A and 7B are representations of another surgical tool of the present invention.

FIG. 8 is a representation of the components of a kit of the present invention.

FIG. 9 is a representation of a spine and surgeon accessing a site of interest using tools of the present invention.

FIG. 10 is a representation of a portion of a soft tissue dilator accessing a site of interest.

FIG. 11 is a representation of a foraminotomy sleeve accessing a site of interest.

FIG. 12 is a representation of a foraminotomy sleeve and stylus within the foraminotomy sleeve, both accessing a site of interest.

The aforementioned figures are provided for illustrative purposes only. The items and elements in the set of figures and within any one figure are not necessarily drawn to scale. Thus, various features may be enlarged relative to other features in order to enable the reader to gain further understanding of the invention.

### DETAILED DESCRIPTION OF THE INVENTION

The present invention provides tools, methods, techniques and kits for use in surgical procedures. Throughout this disclosure, each tool may at times be described as being used in combination with one or more of the other tools described herein or otherwise now known to persons of ordinary skill in the art. The design and use of each tool individually and in combination with one another are within the scope of the present invention.

### Foraminotomy Sleeve

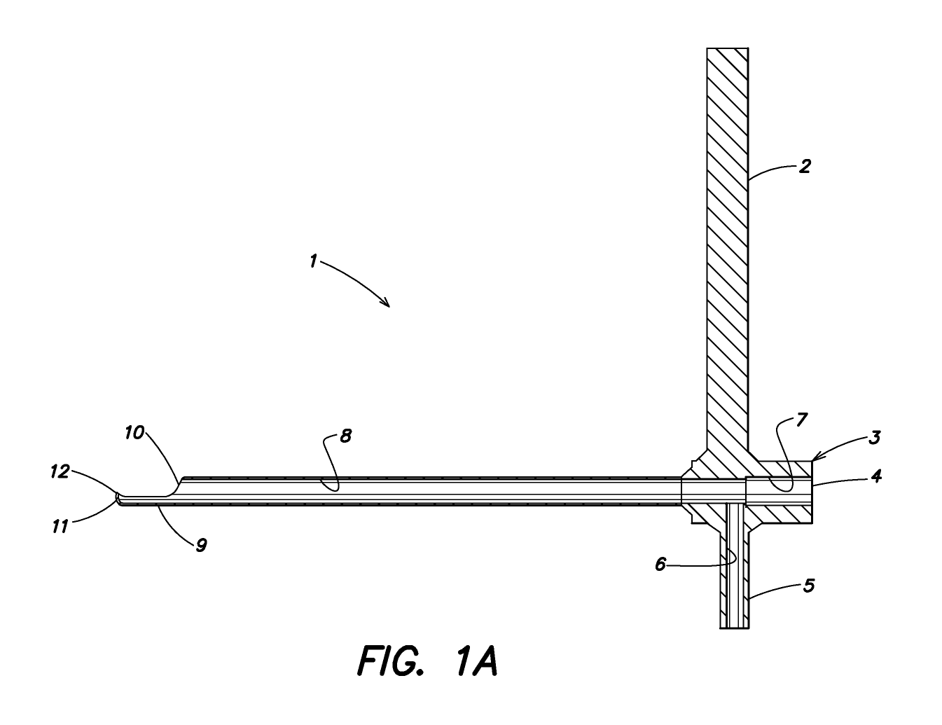

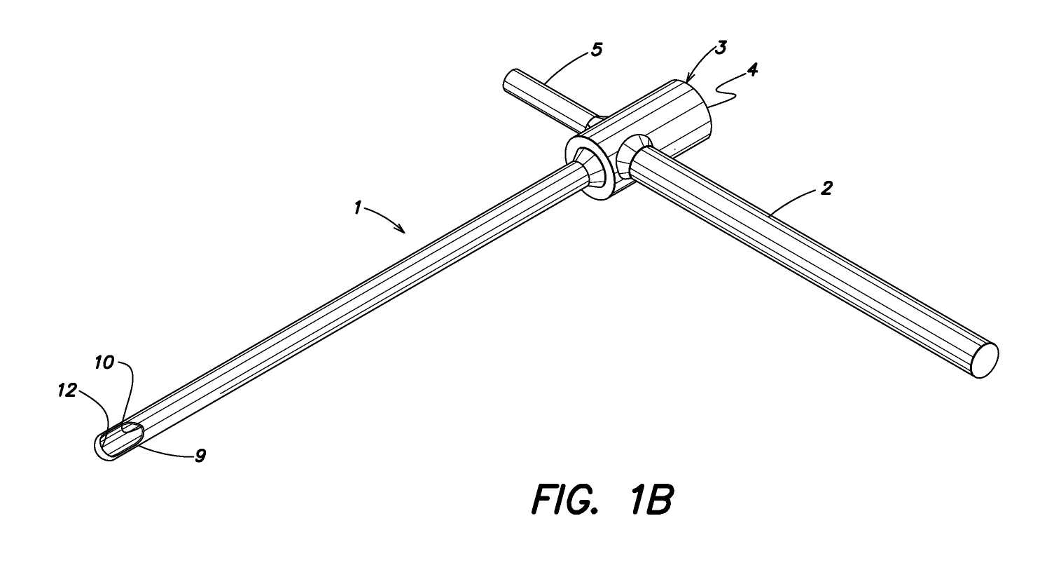

According to a first embodiment, the present invention provides a surgical tool that may be used as a foraminotomy sleeve. Various embodiments of the foraminotomy sleeve may be understood by reference to FIGS. 1A and 1B. FIG. 1A is a cross-section of a foraminotomy tube, whereas FIG. 1B is a three-dimensional representation of a foraminotomy sleeve.

The foraminotomy sleeve 1 provides a housing 8 through which other tools, liquids such as water or saline, and suction may access a site of interest. The sleeve forms a first tube that has a first end and a second end. The tube has an inner passage that runs from the first end 4 to the second end 10. When in use, the first end remains outside of a patient while the second end is within the patient. The walls of the tubes are rigid or semi-rigid and are solid. Consequently, the first tube can remain within the cavity of a body, withstand typical lateral forces while therein, and function as a housing through which to transport material, liquid or other tools to a site of interest.

The first tube may, for example, be a hollow cylindrical, substantially cylindrical or other shape through which materials, or tools may be transported. Thus, it is open on each of the first end and the second end. It may, for example, be 80 mm to 250 mm in length or 100 mm to 200 mm in length or 110 mm to 150 mm in length or 120 mm to 140 mm in length. These lengths include the elongated section discussed in subsequent paragraphs. The cross-section of the tube may be a regular or irregular shape. In some embodiments, the bore, which refers to the inside diameter of the tube, i.e., exclusive to the walls of the tube has a cross-section that is a circle, ellipse or oval. The diameter of the bore (or largest distance when the bore has a cross-section that is not a circle) may, for example, be 3 mm to 10 mm or 4 mm to 8 mm or 5 mm to 7 mm. This bore size, in some embodiments, may be constant throughout the first tube. In other embodiments, it may contain a section with an enlarged diameter at, for example, the first end, which as discussed more fully below, may be within a region that is surrounded by an adapter. The outer diameter of the sleeve that forms the first tube may, for example, be 4 mm to 12 mm or 5 mm to 10 mm or 6 mm to 9 mm. The walls of the first tube may have a thickness of, for example, 0.25 mm to 2 mm or 0.5 mm to 1.5 mm. The shape of the outside surface of the walls may be the same or different as the shape of the inside surface of the walls.

The second end comprises an elongated section 9. As FIG. 1A demonstrates, the elongated section is a continuation of less than all of the walls of the first tube. In some embodiments, less than 80%, less than 70%, less than 60%, less than 50%, less than 40% or less than 30% of the sides are continued. In some embodiments 10% to 40% of the sides are continued. In some embodiments, the elongated section is 10 mm to 25 mm in length or 15 mm to 20 mm in length. The elongated section may run seamlessly from the walls of the first tube, as shown in FIGS. 1A and 1B, and the sides of the first tube that do not extend, may terminate at an angle or curve that runs into the elongated section. As shown in FIGS. 1A and 1B the elongated section may be formed from solid, unbroken material, i.e., there are no holes or spaces.

In some embodiments, the inside of the elongated section may form a cavity. In other embodiments, it may be completely or partially solid. If it forms a cavity, the cavity may with respect to the walls that are extended retain the same shape as the tube that is extended or it may be a different shape.

In some embodiments, the sides of the elongated section may have a height that is 0.5 mm to 4 mm or 1 mm to 3 mm or 1.5 mm to 2.5 mm. This height is the distance from the outside edge of the elongated section that forms the bottom cavity of that section to the top of the walls of that section. In some embodiments this height is from 15% to 80% or 20% to 50% or 20% to 40% or 25% to 35% of the diameter of the outside edge of the first tube. These measurements of the elongated section are exclusive of any knurl, hook or lip that may be present.

The end of the elongated section 9 may be solid 11 and blunt and contain a lip and/or be shaped to form a hook 12 thereby rendering the device to have a concave terminal structure on the inside of the device. The hook may be referred to as a facet hook protective tip. The feature may be configured to hook to a portion of the facet joint, which may be the side that is not located at the site of interest. Thus, it may engage the medial aspect of the facet joint and protect the back side of the facet joint while the practitioner cuts the front side. The outside of the device at the terminus may, for example, be rounded (e.g., convex), flat or a combination thereof. The hook or lip, if present, may have a height of 0.25 mm to 1.5 mm or 0.75 mm to 125 mm as measured from the edge of the elongated section. The lip or hook may, for example, be formed over the last 5% to 25% or 10% to 20% of the elongated section, and it may, by way of non-limiting examples, be designed to have a gradual slope of, for example, 20 to 70 degrees or 30 to 60 degrees.

At the first end of the foraminotomy sleeve and surrounding in part or in the entirety of the outer walls of the first tube at this end may be an adapter 3. It is to the adapter that a handle and a port may be attached, and into which they may seamlessly flow or out of which they may emerge. Thus, the handle and/or port may be part of the adapter or separate structures that are connected to the device through the adapter. Furthermore, as FIGS. 1A and 1B show, the adapter may be structured to completely surround and be attached to a portion of the first tube at the first end, but to leave the opening at the first end unobstructed. The adapter may also contain a body that is tapered at the end distal to the first opening.

The outer diameter of the body of the adapter, which may be a regular or irregular shape, may, for example, be about 10 mm to about 25 mm (exclusive of the port and handle). The first end of the adapter, which may co-terminate at the same location as the first end of the walls of the first tube, may have within it a part of the first tube that has a diameter that is greater than the diameter of the first tube at the second end. When two such diameters exist, they may be referred to as a first diameter and a second diameter, with the first diameter appearing within a region near the first end of the first tube and the second diameter appearing over the first tube. These diameters may, in some embodiments, each be uniform over different ranges of the first tube. In some embodiments the first end of the first tube has a first internal width and the second end of the first tube has a second internal width. The first width may be greater than the second width. For example, over the first about 10 mm to about 20 mm, within the adapter the first tube may contain a region that has a first diameter 7 that is 0.5 mm to 3 mm or 0.75 mm to 2 mm or 1 mm to 1.5 mm larger than the inner diameter of the first tube in the remainder of the adapter and over the remainder of the first tube, where there is the second diameter.

The foraminotomy sleeve may also comprise a port 5. The port comprises a second tube 6 that is connected to the first tube. The connection is configured so that the material can flow between the first tube and the second tube. The port is housed in a port housing structure that abuts from the first tube within the third or quarter of the first tube near the first end of the first tube. In some configurations, the second tube, which is housed within the port housing structure, has an inner diameter that is smaller than the inner diameter of the first tube. When the first tube has regions with two different diameters, the second tube may connect to the first tube in a location that has the smaller diameter or in a location that has the larger diameter. The inner diameter of the second tube may be, about one-half to about three-quarter of the second diameter of the first tube. In some embodiments, it may be 2 mm to 6 mm or 3 mm to 5 mm. As with the first tube, the cross section of the second tube may be any regular or irregular shape such as a circle, ellipse or oval. In some embodiments it is constant.

As noted above, the port housing structure is sized to contain the second tube and may emerge from, be connected to or be part of an adapter that is located at the first end of the device. In some embodiments, the outer surface of the port housing structure may in whole or in part be tapered or it may contain a constant size and thus form a cylinder. The end of the port housing structure distal to the first tube may be sized and configured to permit connection with other devices such as a universal suction house. Thus, by way of example, the port housing structure may house part of or all of (and be coextensive with) a second tube that is 20 mm to 40 mm in length or 25 mm to 35 mm in length. In some embodiments, the portion of the port housing structure closest to the first tube is partially sloped and larger than the remainder of the structure, e.g., the port housing structure has a sloped or partially sloped side wall over 15 mm to 35 mm or 20 mm to 30 mm and in its remainder has a uniform outer diameter of, for example, 5 mm to 10 mm or 6 mm to 8 mm. The center of the port may intersect the first tube 10 mm to 25 mm or 15 mm to 20 mm in from the first end of the first tube.

In some embodiments, the second tube may, for example, join the first tube at a right angle. In other embodiments, the second tube joins the first tube at an angle of less than 90 degrees, for example, 30 to 90 degrees or 45 to 80 degrees relative to the first tube, with the acute angle being between the second tube and the first end of the first tube.

The foraminotomy sleeve may also comprise a handle 2. The handle is configured to enable the surgical tool to be positioned or to be rotated along an axis of the sleeve when an appropriate force is applied.

The handle may be connected to the adapter or emerge from the adapter 145-180 degrees from the port. In some embodiments, it is connected to the first tube 180 degrees from the port. The handle may be solid or hollow, but need not contain a bore or tube. In some embodiments, the handle is a cylinder that is 5 mm to 15 mm or 7 mm to 12 mm in diameter and is 50 mm to 150 mm or 75 mm to 125 mm in length. The handle may be oriented at a right angle relative to the first tube or it may for example be 30 to 90 degrees or 45 to 80 degrees relative to the first tube, with the acute angle being between the handle and the first end of the first tube.

In one non-limiting example the foraminotomy sleeve, the adapter is about 28 mm in length and has a body that is cylindrical or has a substantially uniform outer diameter of about 15 mm. Within the adapter there is an opening at its first end that is about 7 mm in diameter that is the start of a tube that retains this diameter for about 16 mm, beyond which the diameter reduces to about 6 mm, and remains that diameter throughout the remainder of the adapter and the first tube. Emerging from opposite sides of the adapter are a solid handle that is about 10 mm in diameter and extends about 100 mm in length beyond the body of the adapter, and a port in a port housing that extends about 25 mm beyond the body of the adapter. Within the port is a second tube that extends through the adapter to the first tube and intersects the first tube approximately 20 mm away from the first end of the first tube at a right angle. The second tube is approximately 30 mm in length and approximately 7 mm in its outer diameter and the second tube has an inner diameter of approximately 4 mm diameter. Thus, the walls are approximately 1.5 mm thick. Approximately, the last 3 mm of the adapter distal from the first end taper in until a portion of the outside walls of the first tube having a 7 mm diameter is met. This tapering is independent of the inside diameter of the first tube.

The portion of the first tube that emerges from the adapter has a uniform outer diameter of about 7 mm and a uniform inner diameter of about 6 mm, both of which have circular cross-sections. From the first end of the first tube (which is within the adapter and terminates at the same location as the first adapter) to the second end, is approximately 152 mm not including an elongated section. The distance from the tapered end of the adapter to the second end, not including the elongated section is 124 mm.

The elongated section is approximately 16 mm in length and begins with a termination of a part of the first tube and forms an open concave cavity that has a height of approximately 2 mm from the outer edge of the bottom to the top of the side walls formed by it.

At the tip of the cavity (distal to the opening of the first tube) there is a lip that is approximately 1 mm high relative to the walls of the cavity of the elongated section at its highest point. This highest portion is reached over a distance of 3 mm. This total distance from the first end of the first tube to the end of the elongated section is approximately 168 mm.

The foraminotomy sleeve, as well as the other tools of the present invention may be made, for example, out of metal, such as stainless steel, metal alloys, plastics or combinations thereof. In some embodiments the tools are manufactured by molding techniques.

### Stylus

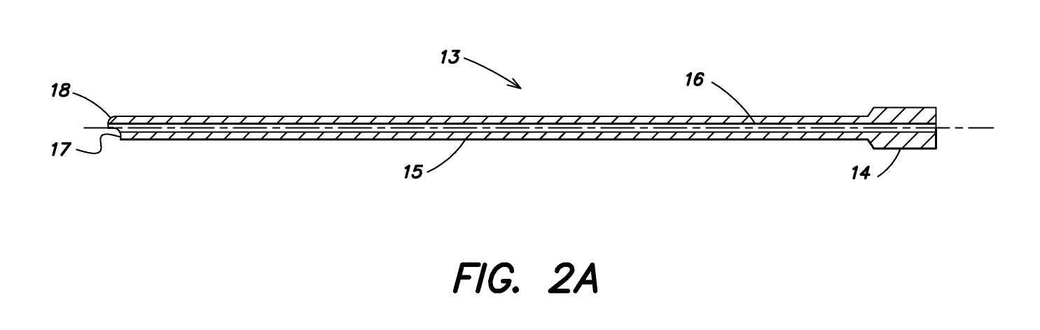

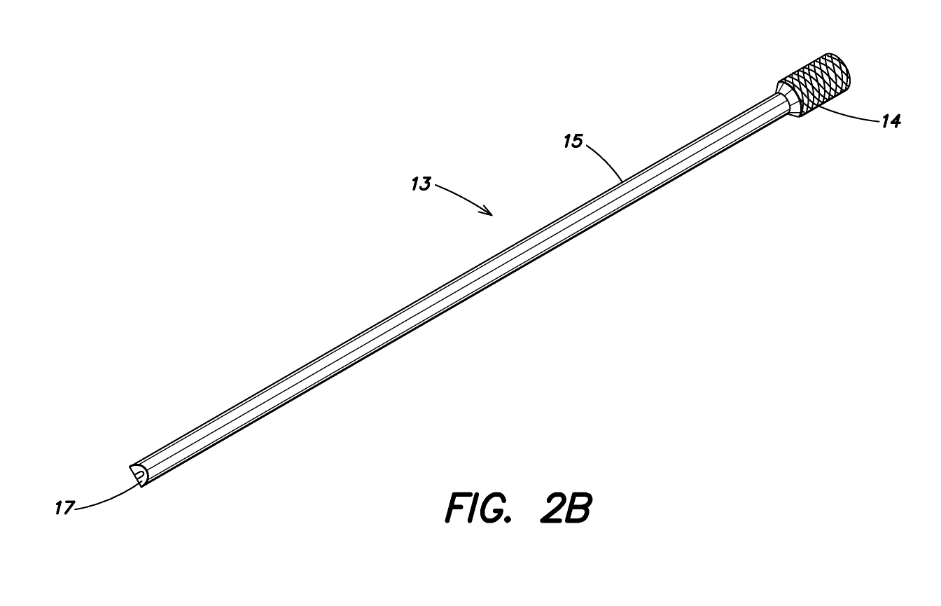

According to another embodiment, the present invention provides a surgical tool that is a stylus. The stylus may be used to push tissue or bone away from a nerve. Various embodiments of the stylus may be appreciated by reference to FIGS. 2A and 2B. FIG. 2A is a cross-section of a stylus, whereas FIG. 2B is a representation of a stylus in three dimensions.

The stylus comprises a tube and an adapter. The tube has a first end and a second end, wherein the second end comprises an elongated section. The elongated section is a continuation of less than all of the sides of the tube. The adapter is located at the first end of the tube and the outer adapter is wider than the tube at the second end.

The stylus 13 may be designed to fit over a guide wire and/or within a foraminotomy sleeve. The guide wire is shown in the broken lines of FIG. 2A and runs along the same dimension as a hypothetical axis. Because the guide wire may be longer than the stylus, it may be advantageous for the stylus' tube to be open at both ends. In FIGS. 2A and 2B, the stylus is shown with an adapter 14. The adapter of the stylus may, in some embodiments, be cylindrical in shape or substantially cylindrical or another regular or irregular shape and have an outer diameter of 5 mm to 20 mm or 8 mm to 15 mm. The length of the adapter may be from 5 mm to 30 mm or 10 mm to 20 mm. The outer surface of the adapter may be smooth, knurled or textured or contain finger grips in order to facilitate gripping by the practitioner who uses it. In one embodiment, the adapter is approximately 15 mm in length and approximately 10 mm in width along its outer diameter. In some embodiments, the adapter is designed to be a size and shape that prevents it from entering the opening at the second end of a foraminotomy sleeve. Thus, its diameter may be larger than that of the opening at the first end of the foraminotomy sleeve.

Spanning the length of the stylus may be a tube 16 that is 100 mm to 300 mm or 150 mm to 250 mm long with openings at both ends. This distance includes the region within the adapter and the elongated section. The inner diameter of the tube (which may have a cross-section that is a circle, an ellipse or an oval or other regular or irregular shape) may, for example, be 1 mm to 3 mm or 1.5 mm to 3 mm. The outer diameter of the longitudinal element exclusive of the adapter may be from 3 mm to 8 mm or from 4 mm to 6 mm. In one embodiment, the diameter of the bore, which has a circular cross section, is approximately 2 mm and the outer diameter of the longitudinal element is approximately 5.5 mm. In some embodiments, the longitudinal element has diameter that is small enough to allow it to fit within the foraminotomy sleeve. Because the stylus may be longer than the foraminotomy sleeve, e.g., 20% to 60% longer or 30% to 40% longer, when in use only a portion of it may be within the foraminotomy sleeve. The walls of the tube 15 not including those covered or formed by the adapter 14 may, for example, be 1 mm to 4 mm or 1.5 mm to 3 mm. The stylus may be designed to nest within the foraminotomy sleeve, and the elongated element of the stylus may nest within the elongated section of the foraminotomy sleeve.

The elongated element 18, which may, for example, be formed over the last 1 mm to 5 mm or 2 mm to 4 mm of the stylus, continues approximately 20% to 80% or 20% to 40% or 30% to 50% of the sides of the longitudinal element 15 beyond where the remainder of the tube ends 17. Additionally, the elongated element of the stylus may form a concave cavity or be solid. In some embodiments, there is neither a terminal lip nor a hook.

In some embodiments, the length of the elongated section is between 0.5% and 5% of the length of the stylus or between 1% and 3% of the length of the stylus.

### Cutter (Foraminotomy Trephine)

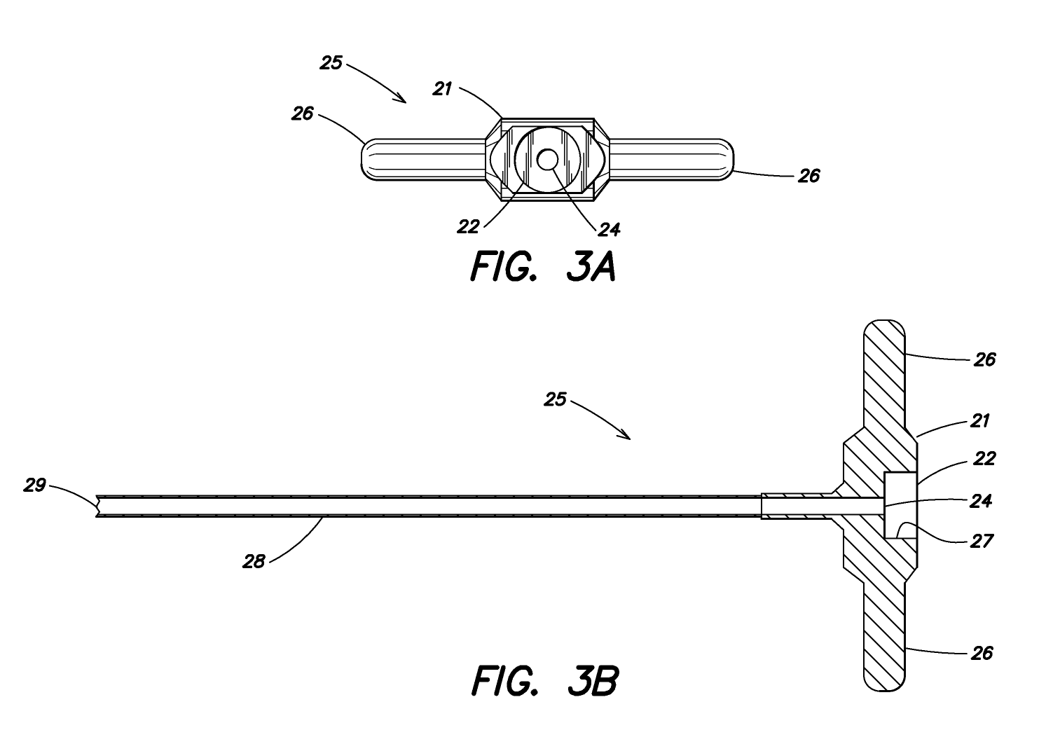

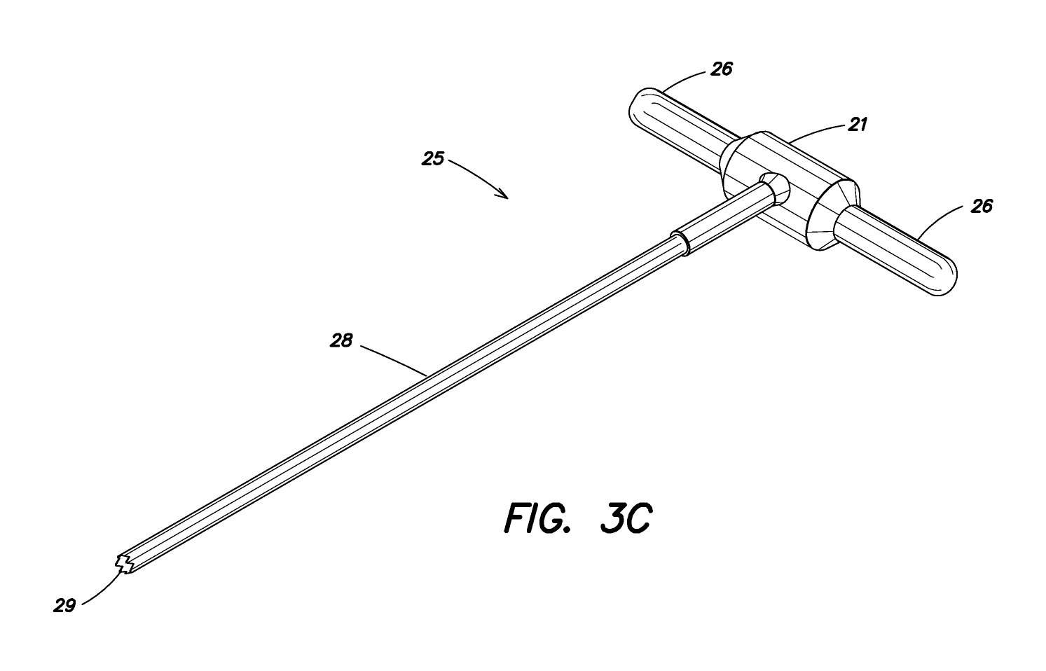

According to another embodiment, the present invention provides a surgical tool that is a cutter. The cutter may be used to separate tissue or bone from other tissue or bone so that pressure may be relieved from the nerve and the offending tissue or bone may be removed. Various embodiments of the cutter may be further understood by reference to FIGS. 3A, 3B and 3C.

The cutter may be designed to fit within a foraminotomy sleeve and the cutter 25 may also be referred to as a foraminotomy trephine. The cutter comprises a tube 28 and a handle 21. The tube has a first end and a second end. The second end may contain a cutting element that may have a blade and for example be serrated 29 or comprise another instrumentality that permits it to cut tissue or bone. The handle is connected to the tube and is configured to cause the surgical tool to rotate when an appropriate force (e.g., clockwise or counterclockwise) is applied.

In some embodiments, the first end of the tube has a first opening 22 and the second end of the tube has a second opening at the serrated edge. The first opening is larger than the second opening. The diameter of the second opening may be the same as the diameter of the bore over the length of most of the tube. This second diameter may exist over the length of the tube until the diameter changes to a first diameter.

The handle 21 may contain one or more gripping members 26 (for example two) that enable the practitioner to use and to manipulate the tool. Within the adapter are two or more regions of the tube with different diameters. In one embodiment, there are only two regions with different diameters. The first region may, for example, have a circular, oval or elliptical cross-section and a diameter of 10 mm to 25 mm or 15 mm to 20 mm. In one embodiment, the cross-section is circular with a diameter that is approximately 16 mm. The transition between the regions may be gradual or abrupt, (i.e., a step).

The second region, which also may have a circular, elliptical or oval cross-section, may, for example, comprise a bore with a diameter of 2 mm to 8 mm or 3 mm or 7 mm. In one embodiment, the cross-section of the bore is circular with a diameter of approximately 5 mm or 4 mm.

The inner diameter of the tube of the longitudinal element may, in some embodiments, be the same as the small diameter of the tube within the handle. The outer diameter of the longitudinal element may, for example by approximately 0.5 mm to 1.5 mm greater than the inner diameter. This additional 0.5 mm to 1.5 mm is the thickness of the walls of the tube. Thus, if the inner diameter is 4 mm, the outer diameter may be 5 mm.

With respect to the handle and by way of a non-limiting example, there may be two gripping elements that are cylindrical and located approximately 180 degrees apart. The gripping elements may each be 5 mm to 20 mm in diameter or 8 mm to 15 mm in diameter, and 15 mm to 45 mm in length or 25 mm to 35 mm in length. In one embodiment, each gripping member is approximately 30 mm in length and 10 mm in diameter. The gripping members may be smooth or textured.

The gripping members may run seamlessly into a central portion of the handle that is from 20 mm to 40 mm or 25 mm to 45 mm in length and 5 mm to 35 mm or 10 mm to 30 mm in width. In one embodiment, the length of the central portion of the adapter is approximately 30 mm (and the length from the tip of one handle to the next is approximately 90 mm) and the width of each handle is approximately 20 mm.

In some embodiments, emerging from the base of the center of the adapter is a reinforcing element that is narrower than the body of the adapter and covers a portion of the outside of the tube over, for example, 10 mm to 30 mm or 15 mm to 25 mm. This reinforcing element may have a cylinder with an outer diameter of 5 mm to 8 mm, e.g., 6 mm.

The entire length of the tube of the cutter may, for example, be 155 mm to 250 mm or 175 mm to 225 mm, e.g., approximately 160.8 mm. In some embodiments the length of the tube that is not covered by the handle or reinforcer is from 60% to 90% of the tube, e.g., approximately 80%.

### Irrigation Coupler

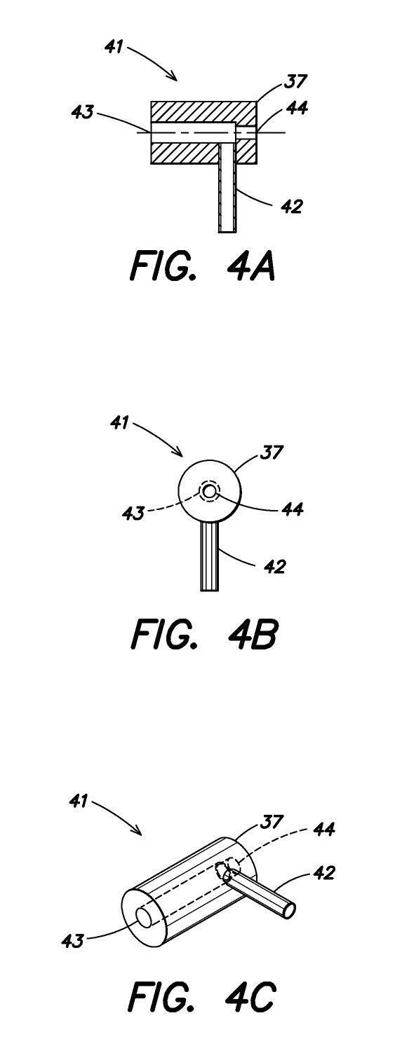

In some embodiments, the surgical tool further comprises an irrigation coupler. The irrigation coupler may be used to provide fluid for irrigation to the site of the interest. Various embodiments of the irrigation coupler can be further understood by reference to FIGS. 4A, 4B and 4C. When in use, a portion of the irrigation coupler may be nested in the first opening of the cutter. The irrigation coupler comprises a tube and a port, wherein a first opening of the tube of the irrigation coupler is located adjacent to the first end of the tube of the surgical tool.

The irrigation coupler 41 may be designed as a cylinder with two openings on opposite ends and a port that intersects a tube that spans from the two ends of the cylinder. In some embodiments, the irrigation coupler's cylinder forms the body 37 of the tool and the irrigation coupler is a size that permits it to nest within or be engaged with the first opening of the cutter. Thus, it may have an outer cross-section that is smaller than or up to the size of the first diameter. In some embodiments, the outer cross-section is less than 2 mm or less than 1 mm smaller than the diameter of the first opening of the cutter. However, the cross-section is preferably greater than the second diameter of the cutter.

By way of non-limiting examples, the outer diameter of the cylinder may be 5 mm to 25 mm or 10 mm to 20 mm. The diameter of the opening at the first end 43 is 2 mm to 5 mm or 3 mm to 4 mm. The diameter of the opening and the opposite end 44 of the cylinder may, for example, be 1 mm to 4 mm or 2 mm to 3 mm. The height of the cylindrical section may, for example, be 15 mm to 35 mm or 20 mm to 30 mm. Thus, when nesting in a cutter, only a portion of the coupler may sit within the tube of the cutter, e.g., 10% to 60% or 20% to 50%.

Within the cylinder, the inner side of the tube may have a first diameter over 60% to 90% of the length of the cylinder or over 70% to 80% of the length of the cylinder, and over the remaining length, have the second smaller diameter.

The port 42 may provide a second tube that extends perpendicularly or at an angle relative to the tube of the cylinder which may be referred to as a first tube. This second tube may connect to the first tube 43 at a location of the larger diameter of the first tube, but within 3 mm or within 2 mm or within 1 mm of where the first diameter ends and the second diameter begins. In some embodiments the first tube and second tube connect at the location where the portion of the first tube has a first diameter.

In some embodiments the port has a constant internal diameter of 1.5 mm to 5 mm or 2 mm to 4 mm and a constant external diameter of 3 mm to 6 mm or 3.5 mm to 5 mm. The length of the port may extend beyond the cylinder of the body by, for example, 4 mm to 10 mm or 5 mm to 8 mm. As persons of ordinary skill in the art will appreciate, the tube within the port extends through the wall of the cylinder so that liquid, suction or material can flow between the first tube and second tube when subjected to the appropriate force.

In one embodiment, the cylinder has a height of approximately 25 mm and a uniform external diameter of approximately 15 mm. The internal diameter of the first opening of this embodiment is approximately 4.1 mm and the internal diameter of the second opening is approximately 3.1 mm. Within the cylinder the bore has the second diameter over approximately 5 mm and the first diameter over approximately 20 mm.

In this example, the port emerges from the body approximately 5.5 mm below the second opening and has an outer diameter of about 4 mm, with an inner bore diameter of about 3 mm.

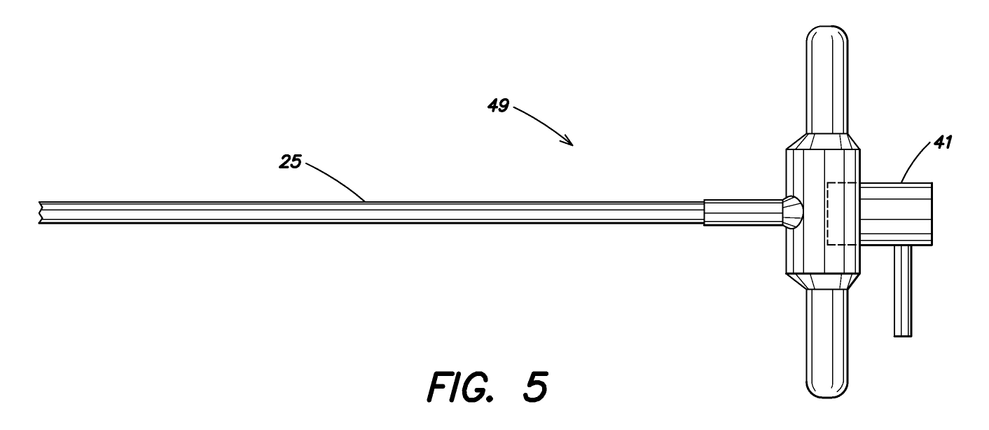

As FIG. 5 shows, the irrigation coupler 41, nests in the first opening of the cutter 25 to form a coupled device 49. The first end of the irrigation coupler is nested within the cutter. In the figures the port is shown parallel to the gripping elements of the cutter. However, that is for illustrative purposes only, and the irrigation coupler can be rotated relative to those handles.

### Soft Tissue Dilator

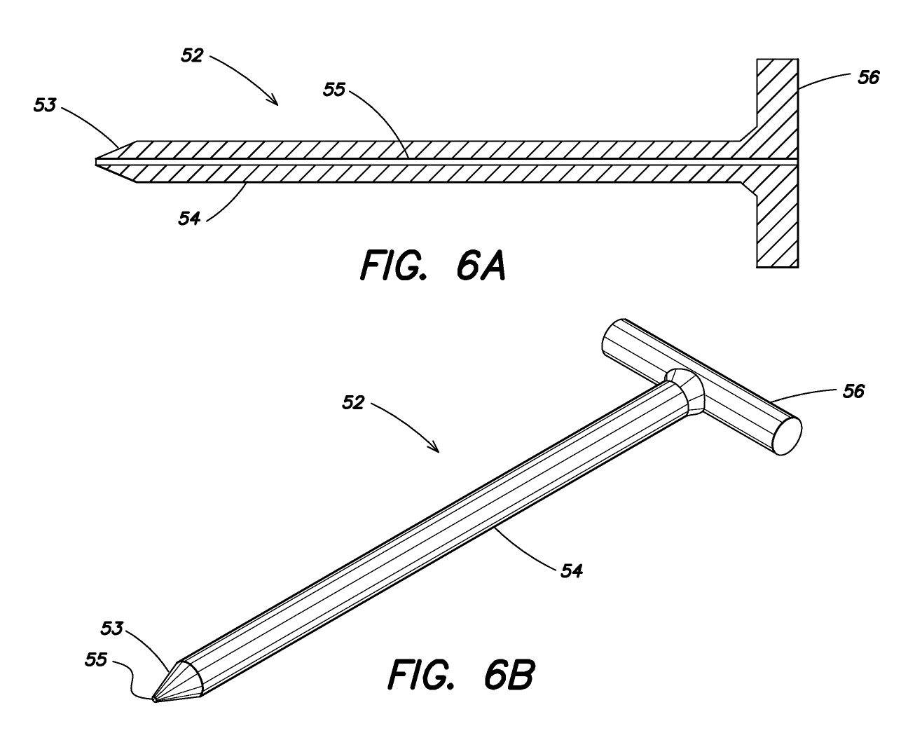

The present invention also provides a surgical tool that is a soft tissue dilator. The soft tissue dilator, which also may be referred to as a cannulated dilator, may be used to enlarge an opening into a patient. Various embodiments of the soft tissue dilator may be understood by reference to FIGS. 6A and 6B.

The soft tissue dilator 52 may be used to create space for easy access by other tools and may comprise a handle 56 and a longitudinal element 54.

The longitudinal element forms a tube 55 that runs along an axis. The handle is located at a first end of the longitudinal element and the tube has at least one opening that is located at a second end of the longitudinal element 53. The outer surface of the tube is tapered at the second end.

In some embodiments the tube spans from the first end of the tube longitudinal element to the second end of the longitudinal element. Preferably, the tube is configured to permit it to fit over a guide wire. Thus, because the guide wire may be longer than the dilator, it may be advantageous to have an opening at both ends of the tube. The inside of the tube may be referred to as a central cannulation. In some embodiments, the tube has a constant diameter that is 0.5 mm to 3 mm or 0.75 mm to 2.5 mm or 1 mm to 1.5 mm. In one embodiment, the tube has a diameter that is approximately 1.06 mm or approximately 1/16 of an inch (or 0.1 mm greater than approximately 1.06 mm or 5/64 of an inch).

The outer diameter of the longitudinal element, which may, e.g., have a circular cross-section, elliptical cross-section or oval cross-section, or other regular or irregular shape may be 15 mm to 35 mm or 20 mm to 30 mm. In one embodiment, it is 25 mm.

The tapering that is located at the second end may, in some embodiments, extend over the outer edge of longitudinal element over 2.5 mm to 20 mm or 5 mm to 15 mm. In one embodiment, the tapering is over a distance of approximately 10 mm at the second end of the longitudinal element.

The handle may be of any configuration that is now known or that comes to be known and a person of ordinary skill in the art would appreciate that may be of use in connection with the present invention. An example of a handle is a T-handle 56. When the handle is a T-handle it may, for example, take the form of a cylinder with a diameter of 5 mm to 20 mm or 8 mm to 15 mm and be a length of 25 mm to 150 mm or 40 mm to 100 mm. In one embodiment, the length is approximately 50 mm.

### Suction Tool

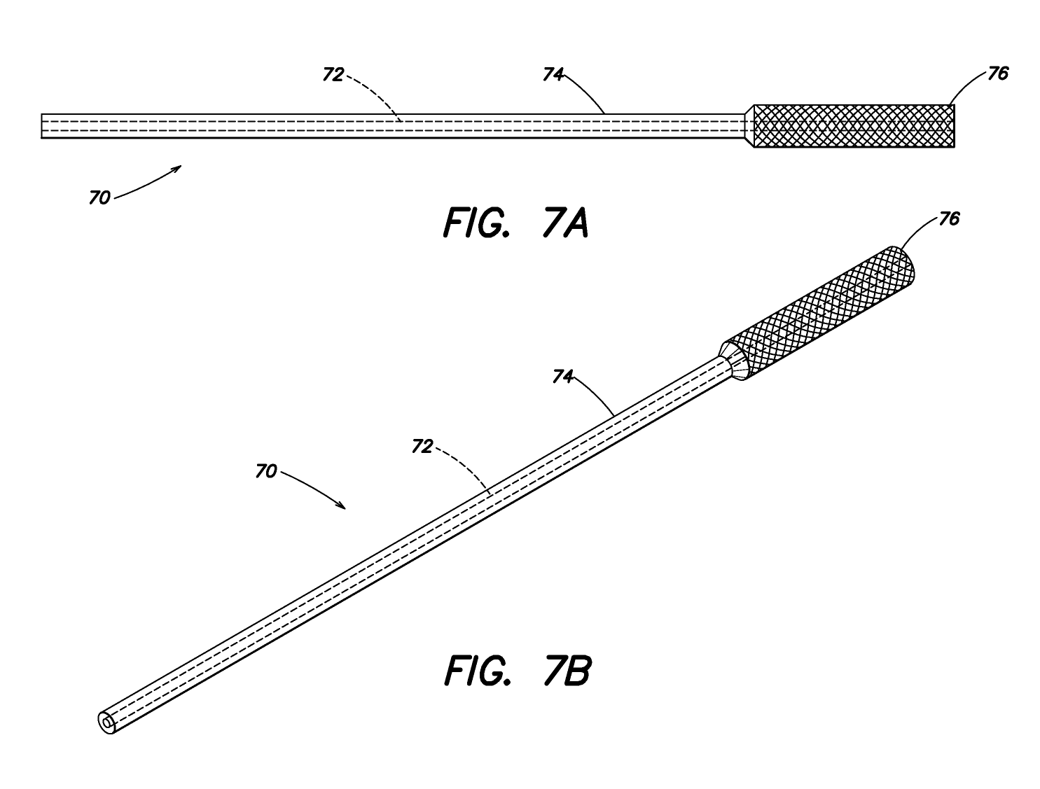

The tools of the present invention may also be used in combination with a suction tool. Various embodiments of this tool may be understood by reference to FIGS. 7A and 7B.

The suction tool may be used after a cutter has been removed. Suction tool 70 has a longitudinal element that forms a tube 72 that spans the length of the longitudinal element 74 and continues within the gripping part 76, which may be knurled. The gripping part is configured to be connected to a second universal suction port. The dimensions of the suction tool may be configured to permit it to fit within the same space as the foraminotomy sleeve.

By way of a non-limiting example, a suction tool may be formed in the shape of a cylinder that is configured to be operably coupled to a wall suction apparatus. The suction tool may, for example, be 175 mm to 225 mm in length (which includes the gripping part), have an inner diameter of 3 mm to 4 mm and outer diameter of 4.5 mm to 6 mm over the majority of the tool, and an outer diameter of 6 mm to 7 mm over the gripping part. The length of the gripping part may be 20 mm to 30 mm. The inner diameter that is located within the gripping part may be the same as throughout the remainder of the cylinder. Thus, in one embodiment the length of the device is approximately 210 mm, the inner diameter throughout the device is approximately 3.5 mm, the outer diameter of the device, exclusive of the portion that corresponds to the gripping part is approximately 5 mm, the outer diameter of the gripping part is approximately 7 mm and the length of the gripping part is approximately 25 mm.

### Kits

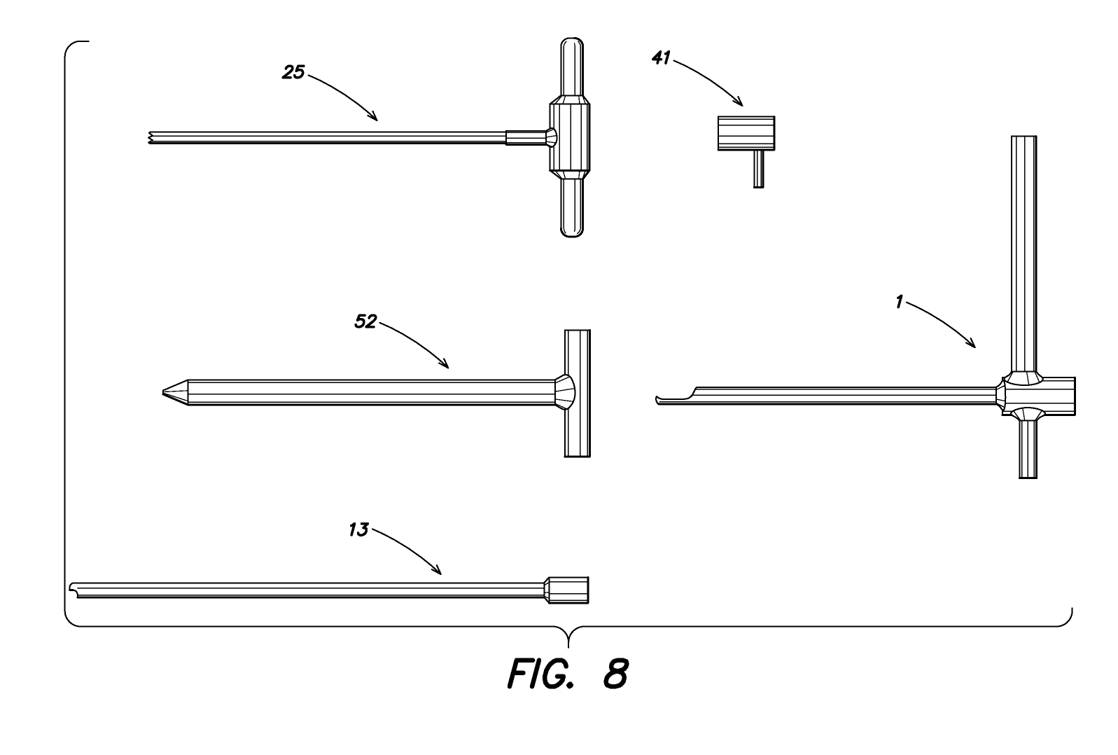

The present invention also provides for kits that may be used in surgeries such as foraminotomies. Examples of kits are those that comprise any of the following: (a) the foraminotomy sleeve and the soft tissue dilator; (b) the foraminotomy sleeve and the stylus; (c) the foraminotomy sleeve and the cutter; (d) the cutter and the stylus; (e) the cutter and the soft tissue dilator; (f) the stylus and the soft tissue dilator; (g) the foraminotomy sleeve, the soft tissue dilator and the cutter; (h) the foraminotomy sleeve, the stylus and the cutter; (i) the foraminotomy sleeve, the soft tissue dilator and the stylus; (j) the soft tissue dilator, the stylus and the cutter; and (k) as shown in FIG. 8 the foraminotomy sleeve 1, the soft tissue dilator 52, the stylus 13 and the cutter 25 with optionally the irrigation coupler 41.

In other embodiments, the present invention provides a kit comprising any of the aforementioned tools or a combination of tools (the kits of the preceding paragraph) and a suction tool as shown in FIGS. 7A and 7B.

### Methods of Use

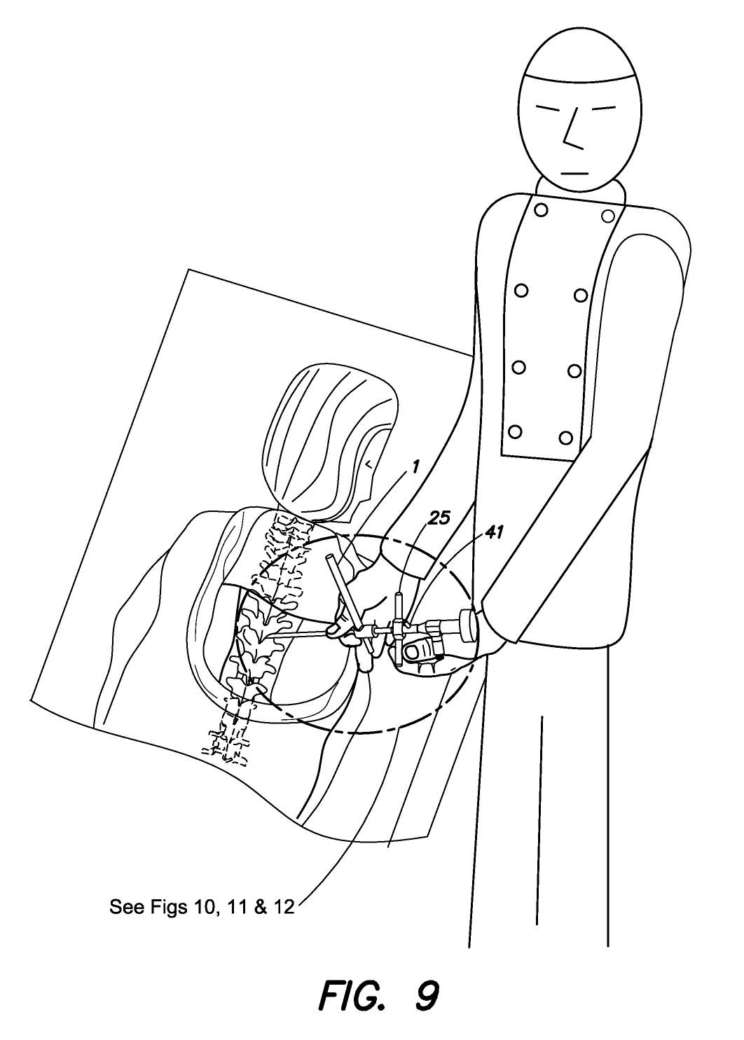

According to a method of the present invention, and as shown in FIG. 9, a patient lies flat. A practitioner first determines where to insert a guide pin. The practitioner may use one or more visualization tools such as x-ray pictures to facilitate locating a site of interest. For example, he may use 1-25 snapshots or 5-20 snapshots or 10-15 snapshots or 12-16 snapshots or 15-20 snapshots.

After the practitioner determines where to start, the practitioner inserts a guide pin. By way of a non-limiting example, the guide pin may be 1/16 of an inch in diameter and between 7 inches and 12 inches in length. A person of ordinary skill in the art will appreciate that the guide pin must be inserted sufficiently far to enable the other tools to be used for their intended purpose, but not so far as to come into contact with and damage tissue. Practitioners will be able to determine when the guide pin has been inserted to a sufficient depth by the resistance they face and/or through the use of visualization tools. Examples of depths to which a guide pin may be inserted are 7 cm to 15 cm from the midline or 9 cm to 11 cm from the midline. A person of ordinary skill in the art will be able to take into account the size of the patient and the location of the site of interest when determining the distance that the guide pin should be inserted.

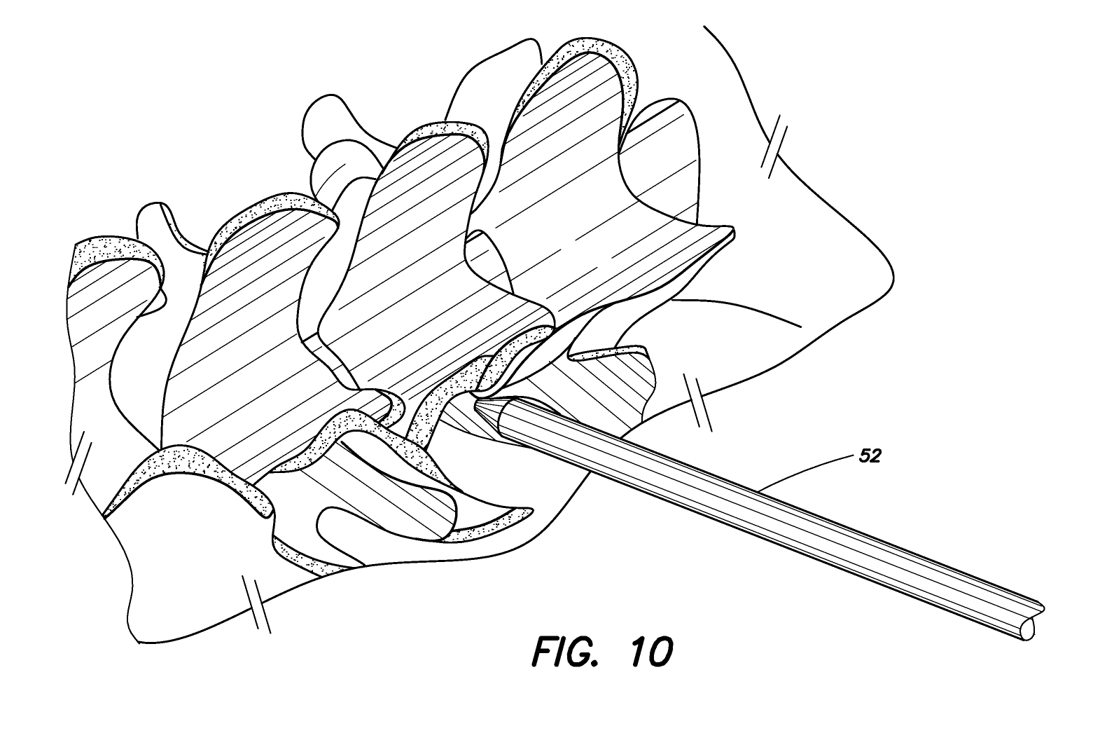

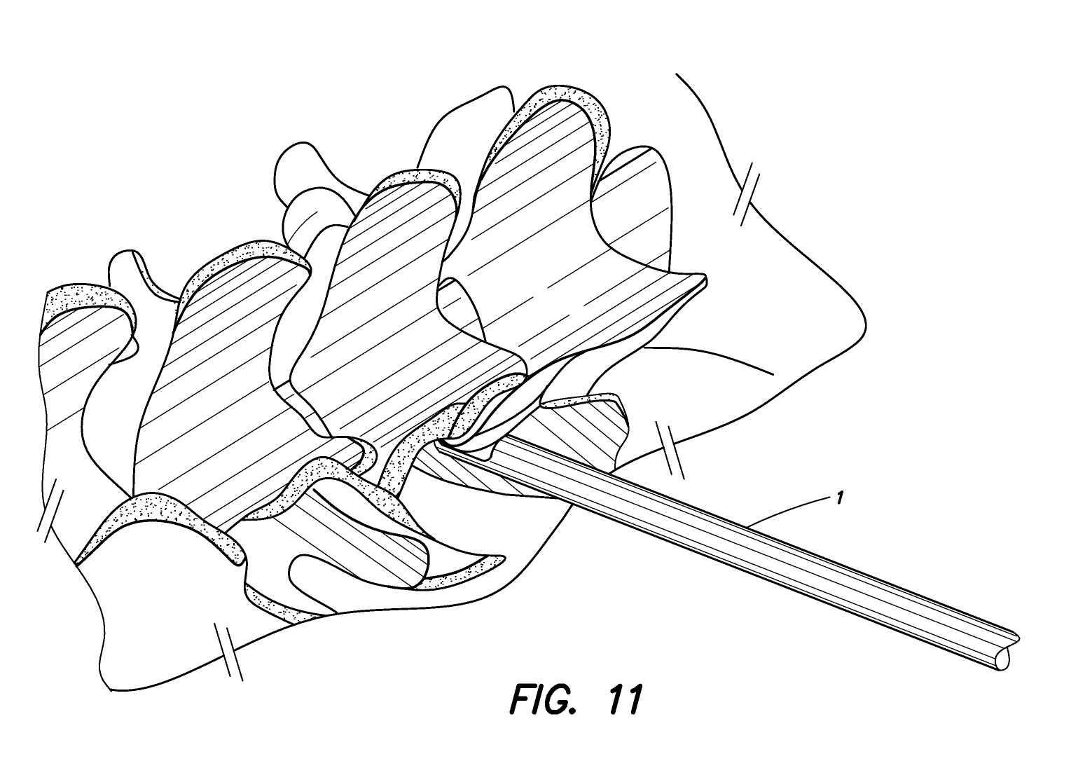

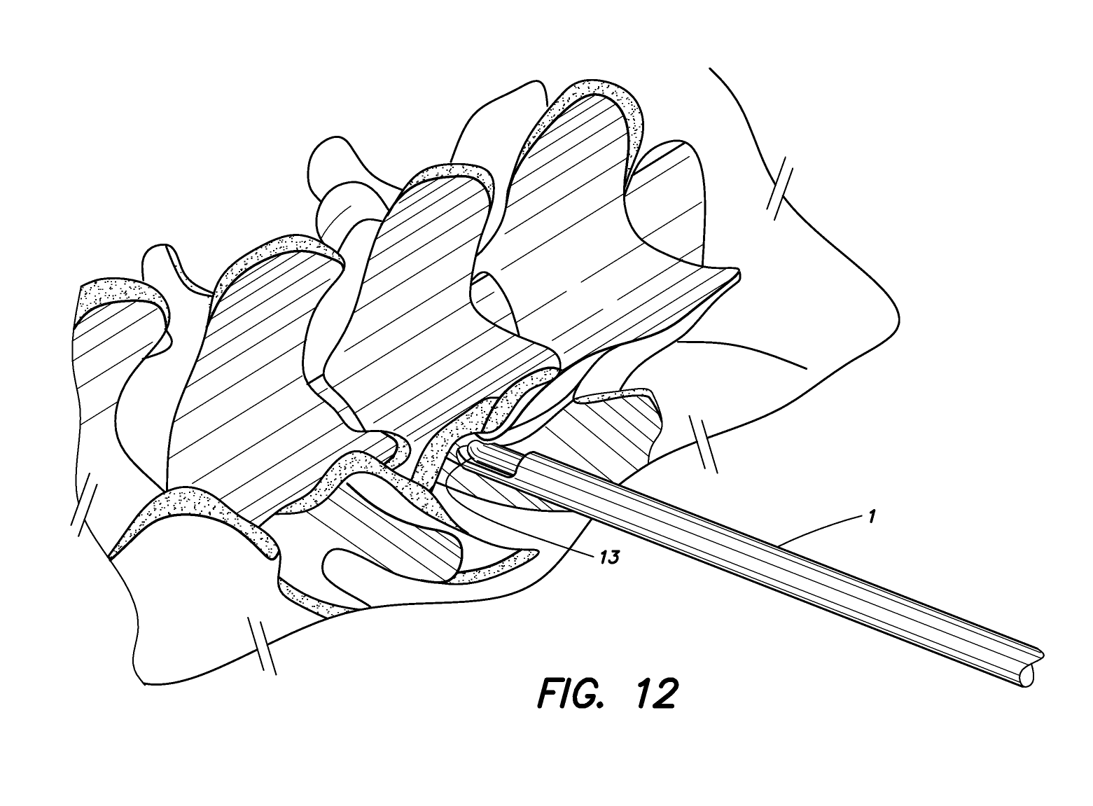

After the guide pin is inserted, the practitioner inserts the soft tissue dilator over the guide pin down to the site of interest. See FIG. 10 showing a soft tissue dilator 52 at a site of interest. The soft tissue dilator pushes tissue away from the guide pin, thereby permitting other tools to be subjected to less resistance when they are inserted. Next, the soft tissue dilator may be removed but the guide pin remains.

After the soft tissue dilator is removed, the foraminotomy sleeve is inserted into the patient over the guide pin, down to the site of interest. See FIG. 11 showing the foraminotomy tube 1 at the site of interest. The tube may come into contact with tissue such as nerve roots, the spinal cord and ligaments, which include the facet joint capsule.

In some embodiments, the foraminotomy sleeve has two arms. The first arm is a directional arm, which when a force is put on it, the foraminotomy sleeve and any tool within the foraminotomy sleeve may rotate along their longitudinal axes, which are aligned with the guide pin. Thus, the directional arm may position the device.

The second arm, which may be shorter than the first arm, may be configured to permit connection between a suction hose (e.g., a universal suction hose) and the inside of the foraminotomy sleeve. The universal suction hose may be part of or configured to be operably coupled with a standard wall suction apparatus. Suction may be used multiple times throughout a process as needed.

Next, the stylus is inserted into the foraminotomy sleeve over the guide pin, but within the foraminotomy tube. See FIG. 13 showing the stylus 13 with the foraminotomy sleeve 1. The stylus and the guide pin are then removed together. The stylus may be used to displace nerve roots and the spinal cord while contacting bone and ligaments such as the facet joint capsule.

After the stylus and the guide pin are removed, the cutter 25 is inserted. The cutter may have a serrated edge. When the cutter is rotated it may cut tissue. Because the elongated section of the foraminotomy sleeve pushes nerves out of the way, the practitioner can more easily and more safely cut tissue that needs to be excised.

Within the foraminotomy sleeve 1 (see FIG. 10) is the cutter 25. The irrigation coupler 41 is inserted into the second end of the cutter. The irrigation coupler is a device through which liquid such as water can be supplied. The endoscope is connected to the irrigation coupler and through this device visualization can be achieved by the use of a camera or scope. In some embodiments, the irrigation coupler and/or cutter comprises or permits the use of a camera such as the Stryker 2.9 Pediatric Endoscope. Liquid may be supplied through the irrigation coupler and down the cutter.

Next, the cutter may be removed to permit further irrigation as needed. Irrigation may, for example, be conducted through the stylus and after the stylus is removed through the use of a surgical syringe. Undesirable material may be removed through the suction port of the foraminotomy sleeve. The cutter may then be put back into the foraminotomy sleeve in order to determine if the desired material was excised. If not, further cutting may be initiated.

Additionally, a practitioner may use one or more instruments in order to facilitate visualization. For example, devices may be used at one or more times during placement of the cutter, during cutting and after cutting, as well as before, during, and after other steps of the methods described herein. They may be used according to techniques and technologies that are now know or that come to be know and that a person of ordinary skill in the art would appreciate would be of use with the present invention.

Unless otherwise specified, any of the features of the various embodiments described herein can be used in conjunction with features described in connection with any other embodiments disclosed. Accordingly, features described in connection with the various or specific embodiments are not to be construed as not suitable in connection with other embodiments disclosed herein unless such exclusivity is explicitly stated or implicit from the context.

### Example

### Prophetic

A patient is placed on an operative table in a prone position. One or more safety straps and monitors are applied. The patient's lumbar spine is then prepped with DuraPrep® and draped in a sterile fashion. A C-arm is also draped. An x-ray image intensification localizes the correct level where the procedure is to be performed on the left or right side as indicated. An oblique view of the facet joint at the appropriate level is visualized. The endplates are squared on the image intensification. The approach is in a midlateral position. The pin, which measures approximately 1/16-inch in diameter, is placed under image intensification to contact the superior articular process at the appropriate level. A one centimeter incision is then made at the pin level where the pin enters the skin. The pin is secured into the superior articular process with gentle tapping on the pin with a mallet.

Next a T-handled soft tissue dilator measuring 6.25 mm in diameter is advanced over the guide pin to create soft tissue access from the skin to the superior articular process. It is then removed.

The cannulated foraminotomy sleeve (with the stylus contained therein) is advanced over the guide pin through the dilated channels through the soft tissues until it contacts the superior articular process. At this point the guide wire is removed. The central cannula stylus is then removed from the foraminotomy sleeve and the sleeve is then advanced under the oblique view toward the ventral direction of the superior articular process until the sleeve enters the foramen at the appropriate level.

During the procedure the patient is slightly sedated, however conscious and able to respond to the surgeon's verbal questioning. At all times the patient maintains verbal confirmation with the surgeon and the anesthesia provider to ensure that the exiting nerve root is in safe position away from the instrumentation.

At this point the image intensifier is readjusted so that an anterior posterior direction view is obtained of the level on the lumbar spine where the procedure is performed. The sleeve at this point is advanced under direct visualization on the image intensifier on the anterior posterior view, while feedback is maintained from the patient to ensure safety of the exiting nerve root. Once the device has reached the appropriate level of lateral to medial advancement, the opening in the foraminotomy sleeve is oriented toward the facet joint. At this point the endoscopy camera is introduced into the sleeve in order to visualize the undersurface of the facet joint where the bone spurs and the narrowing within the foramen is occurring. Once visualization of the anatomy is achieved, the serrated circular T-handled cutting device is introduced into the sleeve and the bone spur is removed. At this point the foraminotomy sleeve is suctioned again in a repeated fashion. With insertion of the scope the removed bone fragments and bone spurs can be visualized. The scope is retracted at this point and the foraminotomy sleeve reoriented to address any additional bone spurs that may have been present at the undersurface of the facet joint. Irrigation and suction cleared the field of any obstructive fragments. The scope is reinserted and the visual confirmation is obtained of the undersurface of the facet joint. Once the bone spurs are visualized within the foraminotomy sleeve, the scope is retracted. The step is repeated with the serrated circular cutting tool to remove the additional bone spurs and possible excess ligaments within the foramen. Irrigation and suction is completed to remove the fragments from the sleeve. Again, reinsertion of the scope confirms that the undersurface of the facet joint has been freed of any protruding bone spurs. Once the procedure is confirmed to be completed, irrigation and suctioning removed any possible fragments that have remained within the sleeve. The foraminotomy sleeve is retracted at this point from the foramen and removed from the patient.

The patient confirms at all times during the procedure with both verbal communication and with movement of the foot in a plantarflexion and dorsiflexion fashion the integrity of the nerve roots and the integrity of the nerve functioning during the procedure and at the end of the procedure as well. Sensation is also tested throughout the procedure with the circulating assistant touching the patient's anatomical areas that would correspond to the exiting nerve root.

I claim:

1. A surgical tool comprising:
(a) a sleeve, wherein the sleeve forms a first tube that has a first end and a second end, wherein the second end comprises an elongated section, wherein the elongated section forms a continuation of less than all of the walls first tube;
(b) a port, wherein the port comprises a second tube that is connected to the first tube; and
(c) a handle.

2. The surgical tool of claim 1, wherein the first end of the first tube has a first width and the second end of the first tube has a second width and wherein the first width is greater than the second width.

3. The surgical tool of claim 1, wherein the elongated section comprises a solid concave terminus.

4. The surgical tool of claim 3, wherein the solid concave terminus comprises a hook.

5. The surgical tool of claim 2, wherein the second tube connects to the first tube where the width of the first tube is the second width.

6. A surgical tool comprising:
(a) a handle; and
(b) a longitudinal element comprising a tube, wherein the handle is located at a first end of the longitudinal element and the tube has at least one opening that is located at a second end of the longitudinal element and wherein an outer surface of the second end of the longitudinal element is tapered.

7. The surgical tool of claim 6, wherein the tube spans from the first end of longitudinal element to the second end of the longitudinal element and has an opening at said second end.

8. The surgical tool of claim 7, wherein the handle is a T-handle.

9. A surgical tool comprising:
(a) a tube, wherein the tube has a first end and a second end, wherein the second end comprises an elongated section, wherein the elongated section is a continuation of less than all of the walls of the tube; and
(b) an adapter, wherein the adapter is located at the first end of the tube and the adapter is wider than the tube.

10. The surgical tool of claim 9, wherein the length of the elongated section is between 0.5% and 5.0% of the length of the surgical tool.

11. A surgical tool comprising:
(a) a tube, wherein the tube has a first end and a second end, wherein the second end comprises a blade or is serrated; and
(b) a handle.

12. The surgical tool of claim 11, wherein the first end of the tube has a first opening and the second end of the tube has a second opening, wherein the first opening is larger than the second opening.

13. The surgical tool of claim 12 further comprising an irrigation coupler, wherein a portion of said irrigation coupler is located in the first opening and the irrigation coupler comprises a tube and a port.

14. A kit comprising a first surgical tool that is the surgical tool of claim 1, and a second surgical tool, wherein the second surgical tool comprises:
(a) a handle; and
(b) a longitudinal element comprising a tube, wherein the handle of the second surgical tool is located at a first end of the longitudinal element and the tube has an opening that is located at a second end of the longitudinal element and wherein an outer surface of the second end of the longitudinal element is tapered.

15. A kit comprising a first surgical tool that is the surgical tool of claim 1 and a second surgical tool, wherein the second surgical tool comprises
(a) a tube, wherein the tube of the second surgical tool has a first end and a second end, wherein the second end comprises an elongated section, wherein the elongated section is a continuation of less than all of the walls of the tube of the second surgical tool; and
(b) an adapter, wherein the adapter is located at the first end of the tube of the second surgical tool and the adapter is wider than the tube of the second surgical tool.

16. A kit comprising a first surgical tool that is the surgical tool of claim 1, and a second surgical tool, wherein the second surgical tool comprises:
(a) a tube, wherein the tube of the second surgical tool has a first end and a second end, wherein the second end comprises a blade or is serrated; and
(b) a handle, wherein the handle of the second surgical tool is connected to the tube of the second surgical tool and is configured to cause the second surgical tool to rotate when a force is applied.

17. The kit of claim 14 further comprising a third surgical tool, wherein the third surgical tool comprises:
(a) a tube, wherein the tube of the third surgical tool has a first end and a second end, wherein the second end comprises an elongated section, wherein the elongated section is a continuation of less than all of walls of the tube of the third surgical tool; and
(b) an adapter, wherein the adapter is located at the first end of the tube of the third surgical tool and the adapter is wider than the tube of the third surgical tool.

18. The kit of claim 15 further comprising a third surgical tool, wherein the third surgical tool comprises:
(a) a tube, wherein the tube of the third surgical tool has a first end and a second end, wherein the second end comprises a blade or is serrated; and
(b) a handle, wherein the handle of the third surgical tool is connected to the tube of the third surgical tool and is configured to cause the third surgical tool to rotate when a force is applied.

19. The kit of claim 18 further comprising a fourth surgical tool, wherein the fourth surgical tool comprises:
(a) a handle; and
(b) a longitudinal element comprising a tube, wherein the handle of the second surgical tool is located at a first end of the longitudinal element and the tube has an opening that is located at a second end of the longitudinal element and wherein an outer surface of the second end of the longitudinal element is tapered.

20. The kit of claim 19 further comprising a suction tool.
